(19) United States
(12) Patent Application Publication
(10) Pub. No.: US 2009/0126134 A1
(43) Pub. Date: May 21, 2009

(54) DENTAL TOOL CLEANER

(76) Inventors: Amy A. Whipple, Olympia, WA (US); Daniel J. Vogl, Logan, UT (US)

Correspondence Address: KELLY LOWRY & KELLEY, LLP, 6320 CANOGA AVENUE, SUITE 1650, WOODLAND HILLS, CA, 91367, US

(21) Appl. No.: 12/270,681

(22) Filed: Nov. 13, 2008

Related U.S. Application Data
(60) Provisional application No. 60/988,480, filed on Nov. 16, 2007.

(51) Int. Cl. B08B 1/00

(52) U.S. Cl. 15/104.94

(57) ABSTRACT

A flexibly resilient porous body for cleaning dental tools is positioned in a receptacle. The receptacle may be conveniently held near a patient's mouth. The porous body includes surface grooves or cuts and is impregnated with an antibacterial solution. A dental tool which has been used to scrape the area around a patient's teeth and gums may be passed through the surface groove or cut in the porous body to remove any material and disinfect the tip of the dental tool.

### BACKGROUND OF THE INVENTION

The present invention is directed to a dental tool cleaner, specifically, a curette or similar scraping and/or picking dental tool. More particularly, the present invention is directed to a flexibly resilient porous body containing an anti-bacterial solution and having facing surfaces configured to clean a dental tool when passed therethrough.

One aspect of most dental visits involves teeth cleaning. The method most often used in cleaning teeth is by scraping or picking of bits of food, plaque and or other material that builds up in and around teeth. This material is often cleaned by using a tool having an edge or a pointed end which is scraped in and around the gum line of the teeth. Such tool is often called a curette.

After a curette is used to scrape or clean around the gum line, it usually has removed food particles, plaque and/or other material adhered thereto. Before the curette can be used to clean another area, the food particles, plaque and/or other material must be removed from the curette. A person using such tool typically has to use a gauze pad or other cloth to remove the material from the tool. Such action requires the use of two hands whereby the dental practitioner would necessarily lose a fulcrum point which may have been established when cleaning a patient's teeth.

In addition, the dental tool may pick up bacteria and/or other microbes when cleaning an area around the patient's gums. It would be beneficial if such bacteria and/or microbes were also cleaned between scrapings rather than being moved from one area of the gum line to another area of the gum line. However, it is difficult to use a gauze pad which has been soaked in an antibacterial. Further, it could become messy or inconvenient to maintain a container of antibacterial solution close by to dip the dental tool in between scrapings. Such a container could not be used between patients, may be wasteful of antibacterial solution, and may be spilled thus creating more work for the dental practitioner.

Accordingly, there is the need for a device whereby a dental practitioner may more easily clean and disinfect a dental tool. The present invention fulfills these needs and provides other related advantages.

### SUMMARY OF THE INVENTION

The present invention is directed to an apparatus for cleaning dental tools. The apparatus comprises a flexibly resilient porous body having a head and a base, and a receptacle in which the base of the porous body is positioned so that the head of the porous body extends therefrom. The head of the porous body contains at least one surface groove or cut and the entire porous body is impregnated with an antibacterial solution.

The porous body preferably comprises a sponge material. The sponge material may comprise a polyester, PVA, low-density polyether, foamed plastic polymer, or medical grade sponge. The sponge material is preferably biodegradable.

The at least one surface groove or cut preferably comprises a plurality of surface grooves or cuts. These surface grooves or cuts should be disposed in the head of the porous body opposite the receptacle.

The receptacle preferably comprises a fluid-impervious material. Further, the receptacle is preferably made from plastic or metal. The anti-bacterial solution preferably comprises chlorhexidine, dextropropoxyphene, or Listerine.

The apparatus preferably includes a means for holding the receptacle while the apparatus for cleaning dental tools is in use. The holding means preferably comprises a spring-biased clip attached to the receptacle, a loop attached to the receptacle for receiving a finger, or a flange associated with the receptacle permitting the flange to be held between two fingers.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the invention. In such drawings:

FIG. 1 illustrates various embodiments of the porous body and the receptacle of the present invention;

FIG. 2 illustrates an exploded perspective view of the present invention;

FIG. 3 illustrates an assembled perspective view of the present invention;

FIG. 4 illustrates a top view of the porous body of the present invention;

FIG. 5 illustrates an environmental view of the present invention;

FIG. 6 illustrates a perspective view of an alternate embodiment of the porous body;

FIG. 7 illustrates a top view of an alternate embodiment of the porous body;

FIG. 8 illustrates an exploded perspective view of an alternate embodiment of the present invention;

FIG. 9 illustrates an assembled perspective view of a preferred embodiment of the present invention;

FIG. 10 illustrates a top view of an alternate embodiment of the porous body;

FIG. 11 illustrates applying antibacterial solution to the porous body of the present invention;

FIG. 12 illustrates the cleaning of a dental tool using the present invention;

### DETAILED DESCRIPTION OF THE PREFERRED EMBODIMENTS

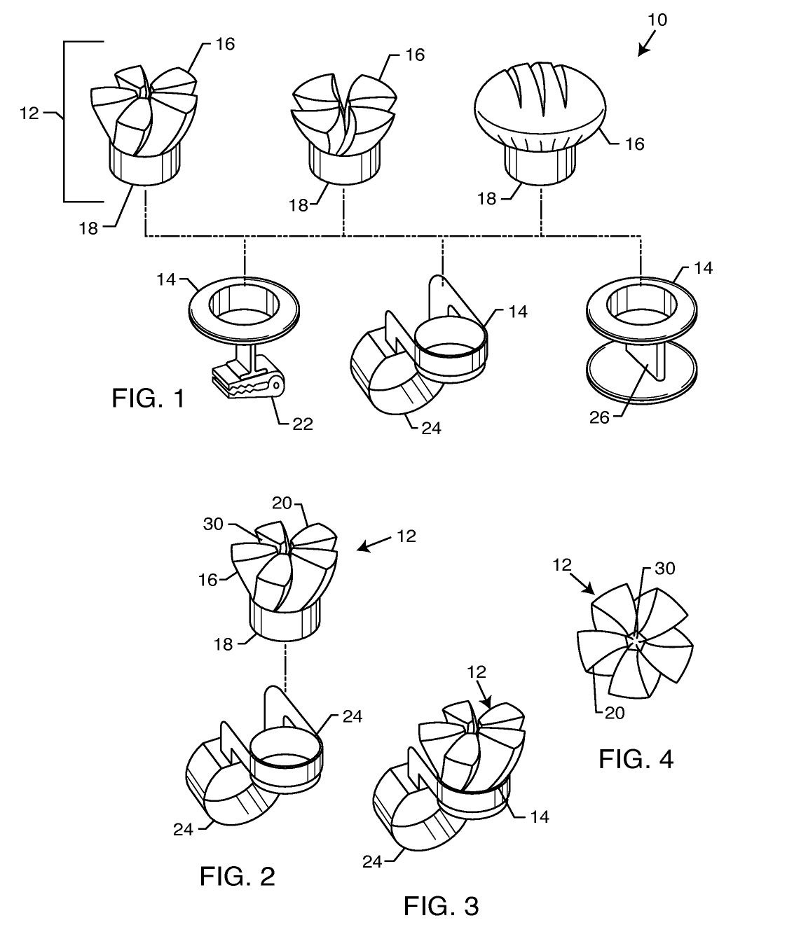

As shown in the exemplary drawings, FIGS. 1 through 12, for purposes of illustration, the present invention is concerned with a novel apparatus for cleaning dental tools generally referred to by reference numeral 10. Different embodiments of the cleaning apparatus 10 will be described below.

As depicted in FIG. 1, the cleaning apparatus 10 generally comprises the porous body 12 and a receptacle 14. The porous body 12 generally has a head 16 and a base 18. The base 18 is generally configured to be positioned in the receptacle 14 such that the head 16 protrudes or extends from the receptacle 14. The porous body 12 is flexibly resilient and preferably comprises a sponge material such as polyester, PVA, low-density polyether, foamed plastic polymer or medical grade sponge. In addition, the porous body 12 is preferably biodegradable.

The head 16 has at least one surface groove 20 therein. The surface groove 20 may be formed in the porous body 12 or created by cutting the material that comprises the porous body 12. Preferably, the head 16 includes a plurality of surface grooves or cuts 20. The surface grooves or cuts 20 are disposed on the head 16 such that they are opposite from the receptacle 14.

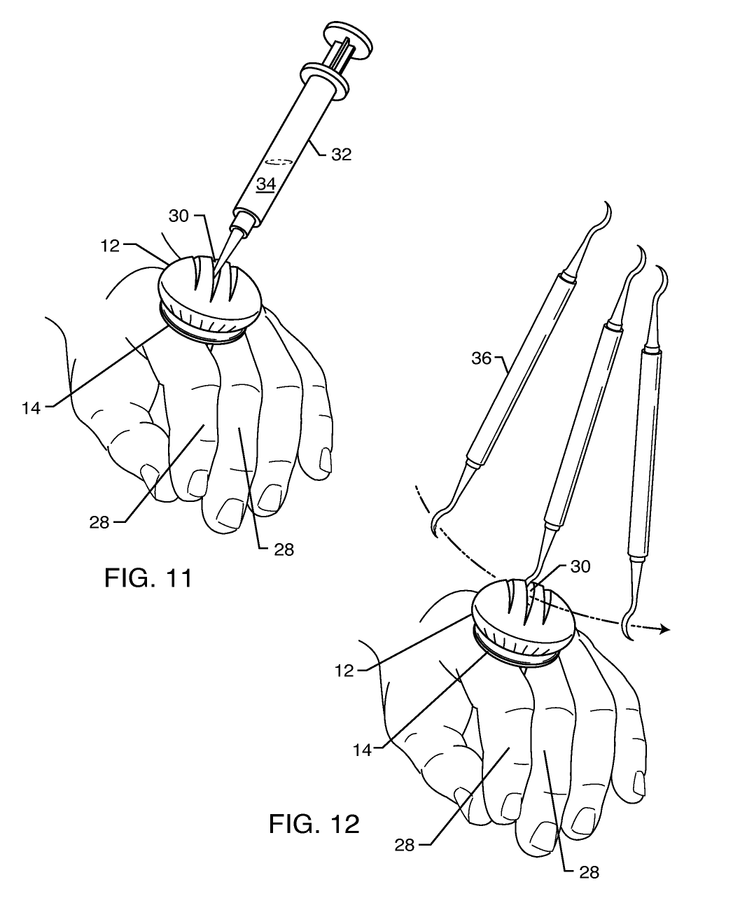

The receptacle 14 preferably comprises a fluid impervious material and may be made from plastic or metal. The receptacle 14 preferably includes a means for holding the receptacle while the apparatus 10 is in use. The holding means may comprise a spring-biased clip 22, a loop 24, or a flange 26 extending away from the receptacle 14. The spring-biased clip 22 may be secured to a surface near a patient's mouth when in use. The loop 24 is preferably configured such that it may receive the finger 28 of a person using a apparatus 10 as illustrated in FIG. 5. The flange 26 is configured such that it may be held between two fingers 28 by a person using the apparatus 10 as illustrated in FIGS. 11 and 12.

FIGS. 2 through 4 illustrate one preferred embodiment of the present invention. In this embodiment, the head 16 of the porous body 12 includes five surface grooves or cuts 20 so as to form multiple exposed surfaces. The surfaces present narrowing areas or valleys 30 where a dental tool may be scraped through to remove debris or other material from the tool. FIGS. 2 and 3 illustrate the assembly of the base 18 of the porous body with the receptacle 14.

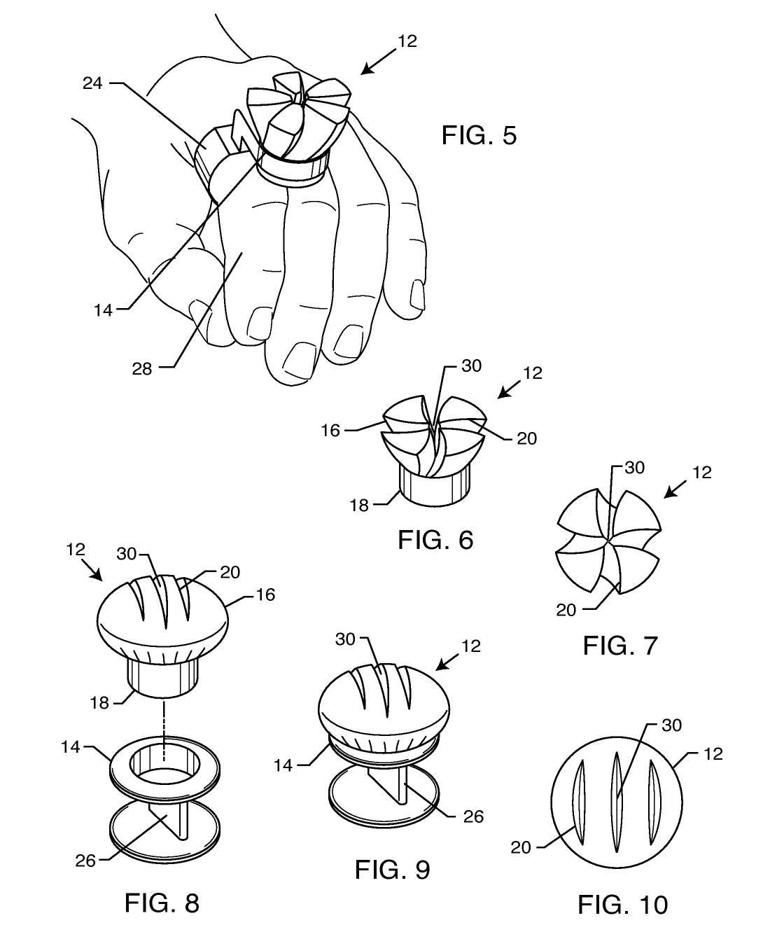

FIGS. 6 and 7 illustrate an alternate embodiment of the porous body 12 of the present invention. In this embodiment, the head 16 includes four surface grooves or cuts 20. These grooves or cuts 20 create narrowing areas or valleys 30 as described above.

FIGS. 8, 9 and 10 illustrate yet a third embodiment of the porous body 12. In this embodiment, the head 16 includes three parallel surface grooves or cuts 20. These grooves or cuts 20 form valleys 30 in the surface of the head 16. FIGS. 8 and 9 illustrate how the base 18 is positioned in the receptacle 14. A person having ordinary skill in the art will appreciate that anyone of the various embodiments of the porous body 12 would be compatible with any one of the various embodiments of the receptacle 14. Accordingly, the illustrated and described configurations of porous bodies 12 with receptacles 14 is not intended to limit the invention.

FIG. 12 illustrates a syringe 32 applying a solution to the porous body 12. The solution is preferably an antibacterial solution 34 which saturates and is absorbed into the porous body 12. The antibacterial solution preferably comprises chlorhexidine, dextropropoxyphene, Listerine or any other known antibacterial/antimicrobial dental solution. The antibacterial solution 34 will destroy microorganisms on the dental tool each time the user passes the tool through the porous body 12 as described below. The fluid impervious nature of the receptacle 14 allows the antibacterial solution 34 to continuously soak the porous body 12 such that the cleaning action remains effective.

As illustrated in FIG. 12, the tip of a dental tool 36 may be passed through the valley 30 in the head 16 of the porous body 12. Passing the dental tool 36 through the valley 30 causes the sidewalls of the valley 30 to wipe the dental tool clean. Any debris or other material on the dental tool 36 is effectively wiped off and retained in the valley 30. Simultaneously, the antibacterial solution 34 cleans any microbes off the dental tool 36. In this way, a dental tool will be cleaned and disinfected between each scraping or picking of a patient's teeth.

Presently, a dentist or dental hygienist typically cleans such dental tools with a cotton square. This action, which requires the use of two hands, causes the dentist or dental hygienist to remove both hands from the patient. Such action not only takes additional time but causes one to lose any fulcrum point that may have been established in holding the patients mouth. In addition, the dentist or dental hygienist runs the risk of sticking his or her fingers with the dental tool through the cotton square. The necessity to use two hands may result in a dentist or dental hygienist not cleaning the dental tool between each tooth that is scraped or picked.

Any of the various holding means described above will free one hand of the dentist or dental hygienist to maintain a fulcrum point when working with the patient's mouth. The dentist or dental hygienist need only move the dental tool 36 from the patient's mouth to the porous body 12 wherever the holding means has the receptacle secured.

Although several embodiments have been described in detail for purposes of illustration, various modifications may be made without departing from the scope and spirit of the invention. Accordingly, the invention is not to be limited, except as by the appended claims.

What is claimed is:

1. An apparatus for cleaning dental tools, comprising:
a flexibly resilient porous body having a head and a base;
a receptacle in which the base of the porous body is positioned so that the head of the porous body extends therefrom;
at least one surface groove or cut in the head of the porous body;
and an antibacterial solution impregnating the porous body.

2. The apparatus of claim 1, wherein the porous body comprises a sponge material.

3. The apparatus of claim 2, wherein the sponge material comprises polyester, PVA, low-density polyether, foamed plastic polymer or medical grade sponge.

4. The apparatus of claim 2, wherein the sponge material is biodegradable.

5. The apparatus of claim 1, wherein the at least one surface groove or cut comprises a plurality of surface grooves or cuts disposed in the head of the porous body opposite the receptacle.

6. The apparatus of claim 1, wherein the receptacle comprises a fluid-impervious material.

7. The apparatus of claim 1, wherein the receptacle is made from plastic or metal.

8. The apparatus of claim 1, wherein the antibacterial solution comprises chlorhexidine, dextropropoxyphene or Listerine®.

9. The apparatus of claim 1, including means for holding the receptacle while the apparatus for cleaning dental tools is in use.

10. The apparatus of claim 9, wherein the holding means comprises a spring-biased clip attached to the receptacle.

11. The apparatus of claim 9, wherein the holding means comprises a loop attached to the receptacle for receiving a finger.

12. The apparatus of claim 9, wherein the holding means comprises a flange associated with the receptacle, permitting the flange to be held between two fingers.

13. An apparatus for cleaning dental tools, comprising:
a flexibly resilient sponge material having a head and a base;
a receptacle in which the base of the sponge material is positioned so that the head of the sponge material extends therefrom;
a plurality of surface grooves or cuts disposed in the head of the sponge material and opposite the receptacle;
an antibacterial solution impregnating the sponge material; and
means for holding the receptacle while the apparatus for cleaning dental tools is in use.

14. The apparatus of claim 13, wherein the sponge material comprises polyester, PVA, low-density polyether, foamed plastic polymer or medical grade sponge.

15. The apparatus of claim 13, wherein the sponge material is biodegradable.

16. The apparatus of claim 13, wherein the receptacle is made from plastic or metal and comprises a fluid-impervious material.

17. The apparatus of claim 13, wherein the antibacterial solution comprises chlorhexidine, dextropropoxyphene or Listerine®.

18. The apparatus of claim 13, wherein the holding means comprises a spring-biased clip attached to the receptacle.

19. The apparatus of claim 13, wherein the holding means comprises a loop attached to the receptacle for receiving a finger.

20. The apparatus of claim 13, wherein the holding means comprises a flange associated with the receptacle, permitting the flange to be held between two fingers.
